(19) United States
(12) Patent Application Publication
(10) Pub. No.: US 2007/0154343 A1
(43) Pub. Date: Jul. 5, 2007

(54) Method and apparatus for deactivating a medical instrument of biocontamination

(75) Inventors: Jude A. Kral, Twinsburg, OH (US); Donald A. Sargent, Wickliffe, OH (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 11/325,191

(22) Filed: Jan. 4, 2006

(51) Int. Cl. A61L 2/04

(52) U.S. Cl. 422/1

(57) ABSTRACT

The present invention provides a medical washer for deactivating surfaces of a medical instrument or device having a lumen. The medical washer includes a housing that defines a chamber dimensioned to contain a liquid and to receive a medical instrument having a lumen. A primary conduit is connected at both ends to the chamber to define a recirculating path for liquid in the chamber. A suction device having an inlet port, an outlet port, and a suction port is disposed within said primary conduit such that the inlet port and the outlet port are fluidly connected to the primary conduit. A suction conduit has a first end that is fluidly connectable to a lumen on a medical instrument and a second end that is fluidly connected to the suction port of the suction device. The suction device is dimensioned such that a flow of the liquid through the suction device creates a negative pressure at the suction port.

### FIELD OF THE INVENTION

The present invention relates generally to microbially deactivating medical instruments, and more particularly, to a system and method for microbially deactivating surfaces of an endoscope.

### BACKGROUND OF THE INVENTION

An endoscope is a medical instrument used for examining hollow organs or body cavities. An endoscope is generally comprised of flexible tubes that contain fiber optic cables that transmit light to illuminate tissue being examined and to return images of the tissue to an eyepiece on the endoscope or to a monitor. Most types of endoscopes also allow for simultaneously obtaining biopsy materials or performing minor surgical procedures. Endoscopes have one or more passages or lumens therein that are exposed to body fluids and tissues during medical procedures. As a result, the endoscope must be washed and microbially decontaminated prior to a subsequent medical procedure to remove any residual body fluids or tissue, conventionally referred to as soil, on surfaces of the endoscope.

A first step in microbially decontaminating the surfaces of an endoscope is to wash the surfaces of the endoscope with a washing chemical to remove soil from the surfaces thereof. This washing is then followed by exposing the surfaces of the endoscope to a microbial decontaminating solution. Heretofore, it was known to wash and/or decontaminate the endoscope by attaching fluid conduits to the various ports on the endoscope and forcing washing fluids and decontaminating fluids under pressure through the endoscope. In such an approach, a separate connection between the inlet port of a lumen and a source of a washing or deactivating fluid is required. As will be appreciated, such an approach is both time-consuming, and subject to human error. Moreover, connecting fittings to the endoscopes creates covered or sealed surfaces that are not exposed to the washing fluids or decontaminating fluids.

Given the importance of deactivating each surface and lumen of an endoscope, there is a need for an effective and direct method for washing and/or microbially decontaminating surfaces of an endoscope.

### SUMMARY OF THE INVENTION

In accordance with one embodiment of the present invention, there is provided a medical washer for deactivating surfaces of a medical instrument or device having a lumen. The medical washer includes a housing that defines a chamber. The chamber is dimensioned to contain a liquid and to receive a medical instrument having a lumen. A primary conduit is connected at both ends to the chamber and defines a recirculating path for liquid in the chamber. A suction device having an inlet port, an outlet port, and a suction port is disposed within the primary conduit such that the inlet port and the outlet port are fluidly connected to the primary conduit. A suction conduit has a first end that is fluidly connectable to a lumen on a medical instrument. The suction conduit also has a second end that is fluidly connected to the suction port of the suction device. The suction device is dimensioned such that a flow of the liquid through the suction device creates a negative pressure at the suction port.

In accordance with yet another embodiment of the present invention, there is provided a system to deactivate biocontamination within a lumen of a medical instrument. The systems includes a tank, a means for holding a medical instrument, a fluid flowpath, a pump, and an eductor. The tank contains a deactivating fluid. Means for holding a medical instrument is dimensioned to hold a medical instrument that has a lumen immersed within the deactivating fluid. The fluid flow path is connected at both ends to the tank to define a closed-loop circulation system. The pump is for conveying the deactivating fluid along the closed-loop path. The eductor 150 is disposed in the closed loop circulation system and has a fluid passage therethrough and a suction port. The eductor 150 creates a negative pressure at the suction port when the deactivating fluid flows through the fluid passage. The system also includes means for connecting the suction port of the eductor 150 to one end of the lumen.

In accordance with yet another embodiment of the present invention, there is provided a method of deactivating biocontamination found within a lumen of a medical instrument. The method includes the steps of: A) pumping a liquid through an inlet of an eductor 150 thereby producing a suction at a suction port of the eductor 150 and, B) drawing a deactivating fluid that surrounds the medical instrument into a lumen inlet port. The suction port of the eductor 150 is fluidly connected to a distal tip of the lumen. The suction produced within the eductor 150 as the liquid is pumped through the inlet port of the eductor 150 draws the deactivating fluid through the lumen and into the eductor 150 through the suction port of the eductor 150.

In accordance with yet another embodiment of the present invention, there is provided a system for microbially deactivating a lumen in a medical instrument. The system includes a housing that defines a chamber for holding a deactivating fluid. The system also includes a closed-loop circulation system for circulating the deactivating fluid in the chamber. The deactivating fluid is withdrawn from the chamber, conveyed along a closed-loop path within the closed-loop circulation system and re-introduced into the chamber. An eductor device is disposed in the closed-loop circulation system and has a fluid passage therethrough. The fluid passage has an inlet end and an outlet end. A flow restriction is disposed within the eductor between the inlet end and the outlet end. A suction port communicates with the fluid passage between the inlet end and the flow restriction. The fluid passage forms a portion of the closed-loop path. The deactivating fluid flows through the eductor from the inlet end to the outlet end when the deactivating fluid is conveyed along the closed-loop path. The flow restriction in the eductor is dimensioned such that flow of deactivating fluid through the eductor creates a negative pressure at the suction port. A suction conduit is connected at one end to the chamber and connectable at another end to a lumen in a medical instrument disposed in the chamber.

In accordance with still another embodiment of the present invention, there is provided a method for microbially deactivating a lumen in a medical instrument. The method includes the following steps, A) providing a closed loop path that is connected at both ends to a chamber for containing a deactivating fluid; B) disposing a medical device having a lumen in the chamber such that an end of the lumen is fluidly connected to the closed loop circulation system; C) conveying a first stream of the deactivating fluid along the closed-loop path, such that the first stream of the deactivating fluid creates a negative pressure within a first portion of the closed-loop path; D) drawing a second stream of the deactivating fluid along a second path from the chamber such that the deactivating fluid is conveyed through the lumen, wherein the second path joins the closed-loop path at the first portion of the closed-loop path; E) combining the first stream of deactivating fluid and the second stream of deactivating fluid at the first portion of the closed-loop path to form a combined stream of the deactivating fluid; and F) reintroducing the combined stream of deactivating fluid into the chamber.

One advantage of the present invention is a method and system for washing and/or microbially decontaminating a medical instrument having a lumen.

Another advantage of the present invention is a system as described above that does not require connecting each endoscope inlet port to a source of washing or decontaminating fluid.

Yet another advantage of the present invention is a method and system as described above wherein a washing or decontaminating fluid is drawn through the lumen.

Another advantage of the present invention is a system as described above that allows an operator to control the rate of flow of a washing or deactivating fluid through a lumen of a medical instrument.

Another advantage of the present invention is a system as described above that allows an operator to control the pressure of a washing or deactivating fluid within a lumen of a medical instrument.

Another advantage of the present invention is a method of washing and/or deactivating that reduces the impact that entrained gaseous bubbles have on washing or decontaminating a lumen by providing a means for controlling the flow rate and pressure of a washing or deactivating fluid within a lumen.

Yet another advantage of the present invention is a system and method as described above that is capable of decontaminating one or more lumens of a medical instrument in a single operation.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a pictorial illustration schematically showing a system capable of cleaning and/or microbially decontaminating medical instruments, according to a preferred embodiment of the present invention;

FIG. 2 is a cross-sectional view of an eductor that forms a part of the system shown in FIG. 1;

FIG. 3 is a cross-sectional view of a free end of a lumen, showing a suction attachment connected thereto; and

FIG. 4 is a graph of pressures and flow rates versus time for water moving through a lumen of a medical instrument.

### DETAILED DESCRIPTION OF PREFERRED EMBODIMENT

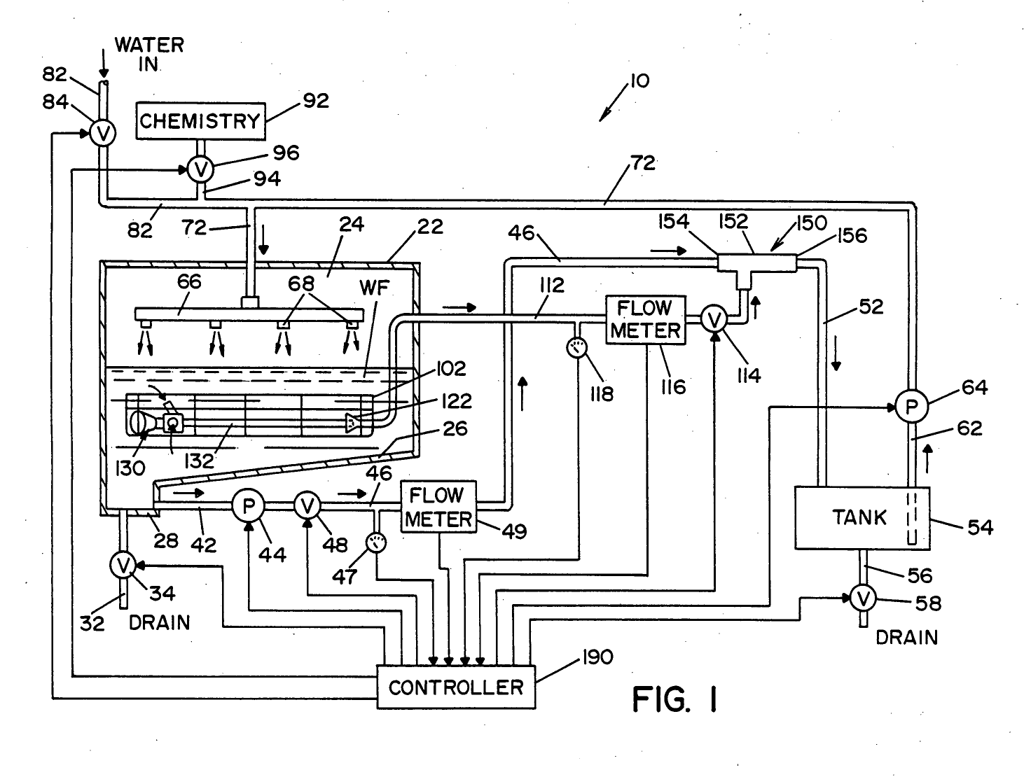

Referring now to the drawings wherein the showings are for the purpose of illustrating a preferred embodiment of the invention only, and not for the purpose of limiting same, FIG. 1 shows a system 10 that may be used for washing and/or microbially decontaminating medical instruments and equipment having passages or lumens therethrough, such as an endoscope. System 10 includes a housing 22 that defines a washing chamber 24. Housing 22 has a bottom wall 26 that is formed to slope toward a sump assembly 28 that is disposed at the bottom of washing chamber 24. As will be described in greater detail below, washing chamber 24 is dimensioned to receive washing fluids, decontaminating fluids and/or rinsing fluids that are used within the washing chamber 24. A drain line 32 communicates with sump assembly 28. A valve 34 is disposed in drain line 32 to control draining of fluids from washing chamber 24. A first conduit 42 connects sump assembly 28 to an inlet of a fluid pump 44 that is driven by a motor (not shown). A second conduit 46 connects an outlet of pump 44 to an inlet end of an eductor 150, that shall be described in greater detail below. A valve 48 is disposed within second conduit 46 between pump 44 and eductor 150 to control the flow of fluid to eductor 150. A pressure sensor 47 and a flow meter 49 are disposed within second conduit 46 between pump 44 and eductor 150 to monitor the pressure and flow rates of fluid to eductor 150. A third fluid conduit 52 connects an outlet end of eductor 150 to a tank 54 that defines a reservoir for holding a washing fluid, a decontaminating fluid and/or a rinsing fluid. A tank drain line 56 extends from tank 54 to allow fluid to be drained therefrom. A valve 58 is disposed within tank drain line 56 to control the flow of fluid therethrough. A fourth conduit 62 extends from tank 54 and connects to an inlet of a second fluid pump 64 that is driven by a motor (not shown). The outlet of fluid pump 64 is connected to a manifold 66 having spaced-apart nozzles 68 within housing 22 by fifth fluid conduit 72. In the embodiment shown, manifold 66 and nozzles 68 are disposed in the upper portion of housing 22 to direct fluid downwardly toward bottom wall 26 and sump assembly 28. As shown in FIG. 1, a water inlet line 82 is in fluid communication with fifth conduit 72. Water inlet line 82 is connectable to a source of water (not shown) for filling washing chamber 24 of housing 22. A valve 84 is disposed within water inlet line 82 to control the flow of water to housing 22. A chemistry source 92, schematically illustrated in FIG. 1, is connected to water inlet line 82 by a conduit 94 to enable chemicals to be added to the incoming water to form a washing fluid or decontaminating fluid. A valve 96 is disposed within conduit 94 to chemical source 92 to control the flow of chemicals introduced into water line 82.

A holder 102 is provided in chamber 24 of housing 22 for holding a medical instrument 130 to be washed or microbially decontaminated. Medical instrument 130 has a tubular portion 132 having a lumen defined therethrough. In the embodiment shown, holder 102 is in the form of a wire rack. Holder 102 is disposed near the bottom of chamber 24, as illustrated in FIG. 1. A suction conduit 112 is provided to extend from chamber 24 to eductor 150. A valve 114 is provided in suction conduit 112 to control flow therethrough. A flow meter 116 and a pressure gauge 118 are provided in suction conduit 112 to monitor the pressure level and flow of fluid therethrough. Connecting means 122 is provided on a free end of suction conduit 112 for connection to tubular portion 132 of medical instrument 130 within chamber 24, as shown in FIG. 3. In the embodiment shown, connecting means 122 is in the form of a conical-shaped member 124 that is dimensioned to abut the free end of tubular portion 132 of medical instrument 130. In this respect, conical-shaped member 124 is dimensioned to seal the end of tubular portion 132 of medical instrument 130 and to simultaneously minimize surface contact between conical-shaped member 124 and the surface of tubular portion 132 of medical instrument 130. As will be appreciated, the conical-shaped member 124 allows tubular portions 132 of different diameter to be attached to suction conduit 112.

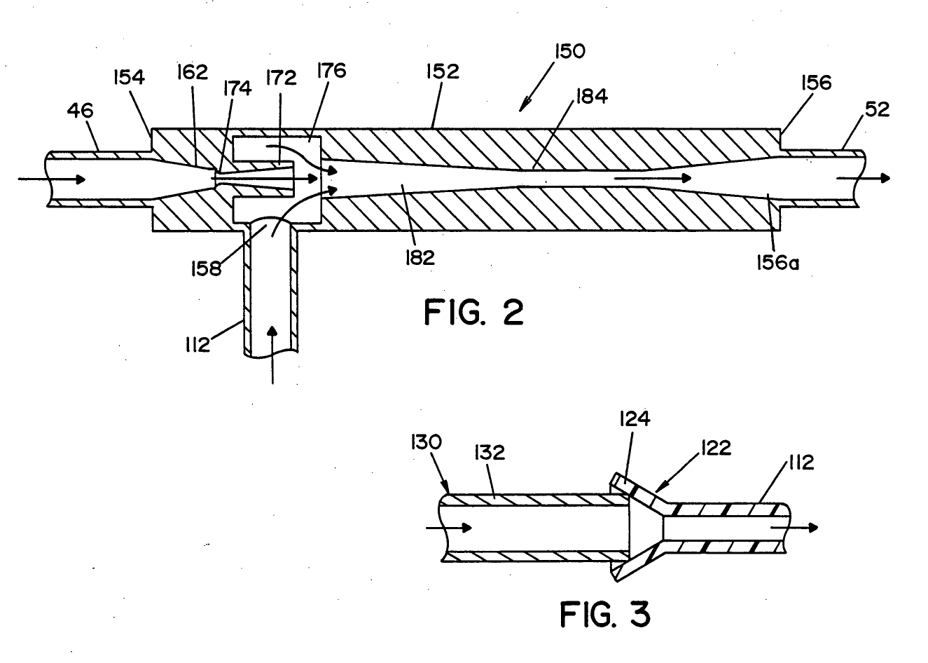

Referring now to FIG. 2, eductor 150 is best seen. Eductor 150 has an elongated body 152, with an inlet end 154, an outlet end 156 and a suction port 158. A continuous passageway 162 extends through elongated body 152 from inlet end 154 to outlet end 156. Adjacent to inlet end 154, a nozzle 172 is formed. Nozzle 172 is formed by tapering passageway 162 at inlet end 154 to form a restriction 174. Restriction 174 flares out at nozzle 172 that is disposed within a mixing chamber 176. Mixing chamber 176 is in fluid communication with suction port 158. An elongated opening 182 extends from mixing chamber 176 to outlet port 156a at outlet end 156 of elongated body 152. Opening 182 tapers down to an area of reduced diameter at throat 184. In this respect, throat 184 and opening 182 are in the form of a Venturi. As will be described in greater detail below, the flow of fluid from inlet end 154 to outlet end 156 of eductor 150 creates a low pressure at the leading end of opening 182, thereby producing a lower pressure at mixing chamber 176 and at suction port 158.

A controller 190 is provided to control the operation of system 10. As schematically illustrated in FIG. 1, control 190 receives input information from the pressure sensors 47, 118 and flow meters 49, 116 in second conduit 46 and suction conduit 112, and provides output control signals to the valves 34, 48, 58, 84, 96, and 114 and to the motors controlling the pumps 44, 64. Controller 190 also includes a pre-inputted program for operating a washing cycle and/or a microbially decontamination cycle that may include one or more initial rinse cycles, a washing for a microbial decontamination cycle and one or more rinse cycles.

Referring now to the operation of system 10, a washing phase of a washing cycle shall be described. Medical instrument(s) 130 to be cleaned are placed within holder 102 within chamber 24 of housing 22. Connecting means 122 of suction conduit 112 is attached to the free end of tubular portion 132 of medical instrument 130, as pictorially illustrated in FIG. 3. With medical instrument 130 in place within chamber 24 of housing 22, controller 190 causes valve 84 in water inlet line 82 to open to allow water to enter chamber 24 via manifold 66 and nozzles 68. Controller 190 also causes valve 96 in chemistry conduit 94 to open to introduce chemistry into the incoming water to produce a washing fluid. During the initial portion of a washing phase, valve 34 in drain line 13 is closed, and pumps 44 and 64 do not operate. As a result, the incoming washing fluid, designated “WF,” fills the lower portion of chamber 24, as illustrated in FIG. 1. According to the present invention, washing fluid WF fills chamber 24 until medical instrument(s) 130 is (are) totally immersed in washing fluid WF, as illustrated in FIG. 1. When washing fluid WF within chamber 24 has reached a desired level, controller 190 activates pump 44 to convey washing fluid WF in sump assembly 28 of housing 22 through first and second conduit 42, 46 to inlet end 154 of eductor 150. Washing fluid WF flows through eductor 150 to tank 54. Once a predetermined level of washing fluid WF is established in tank 54, pump 64, downstream from tank 54, is activated to cause washing fluid WF to be conveyed through the fifth conduit 72, through manifold 66 and back into chamber 24 through spray nozzles 68. Valve 84 in water inlet line 82 is closed. A closed loop fluid flow circuit is thus formed as washing fluid WF flows from sump assembly 28 in housing 22 through conduit circuits 42, 46, 52, 62, and 72 and back into chamber 24 in housing 22 through nozzles 68 in manifold 66.

Washing fluid WF enters eductor 150 at inlet end 154 at a predetermined pressure, hereinafter referred to as the “motive pressure.” The flow rate of washing fluid WF entering inlet port 154 of eductor 150 is hereinafter referred to as the “motive flow rate.” As washing fluid WF is conveyed into eductor 150 and through nozzle 172 of eductor 150, a partial vacuum (Venturi effect) is created at suction port 158 of eductor 150. As used herein, the pressure at suction port 158 of eductor 150 is hereinafter referred to as the “suction pressure.” The lower pressure, i.e., the vacuum, created at suction port 158, draws washing fluid WF within the chamber 24 of the housing 22 through the medical instrument 130. Washing fluid WF is thus drawn through medical instrument 130 and through the lumen of tubular portion 132 into suction conduit 112. The fluid flow rate of washing fluid WF entering suction port 158 is called the “suction flow rate.” Washing fluid WF entering eductor 150 through suction port 158 combines with washing fluid WF flowing through eductor 150 from inlet end 154 to outlet end 156.

Controller 190 monitors the motive pressure, as indicated by pressure sensor 47 within second conduit 46, and monitors the motive flow rate by monitoring flow meter 49 within second conduit 46. Likewise, controller 190 monitors the suction pressure and suction flow rate by monitoring pressure sensor 118 and flow meter 116 within suction conduit 112. Controller 190 can establish a desired suction pressure within suction line 112 by controlling the motive flow rate and motive pressure to eductor 150. In this respect, controller 190 can adjust the output of pump 44 and the position of valve 48 to create a desired motive pressure and motive flow rate to eductor 150. In addition, controller 190 can vary the output of pump 64 that is connected to tank 54 to maintain a desired reservoir of washing fluid within tank 54 and to ensure continuous, circulating flow through system 10.

The suction pressure created at the tip end of the medical instrument 130 creates a flow of washing fluid WF through the lumen of tubular portion 132 of medical instrument 130, thus exposing all internal surfaces of the lumen to washing fluid WF. The operation of system 10 has heretofore been described with respect to a washing fluid WF during a washing phase of a washing cycle. As will be appreciated, during an initial rinse phase or a post washing rinse phase, system 10 operates in a similar manner, but without chemistry being added to the incoming water that enters housing 22 through water inlet conduit 82.

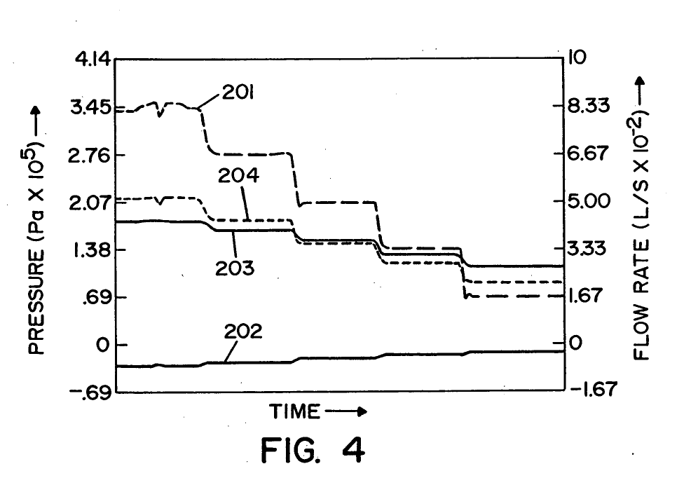

FIG. 4 illustrates how controller 190 can control the motive pressure, motive flow, suction pressure and suction flow. In FIG. 4, a test is conducted where suction conduit 112 is merely placed within a body of water such that connecting means 122 of suction conduit 112 is immersed within the water. Water is forced through eductor 150 to create suction at suction port 158. Trace line 201 of FIG. 4 illustrates various modes of pressure as a function of time. Trace lines 202, 203 and 204 illustrate corresponding suction pressures, motive flow rates and suction flow rates, respectively. Table I provides selective data points that correspond to the trace lines of FIG. 4.

TABLE I

| MOTIVE | MOTIVE | | SUCTION |
|---|---|---|---|
| PRESSURE | FLOW RATE | SUCTION | FLOW |
| (×10⁵ Pascals- | (×10⁻² liters/ | PRESSURE | (×10⁻² liters/ |
| gauge) | second) | (×10⁴ Pascals-gauge) | second) |
| 3.46 | 4.35 | −3.0 | 5.13 |
| 2.76 | 4.05 | −2.4 | 4.37 |
| 2.07 | 3.63 | −2.0 | 3.60 |
| 1.39 | 3.17 | −1.6 | 2.85 |
| 0.70 | 2.73 | −1.3 | 2.17 |

TABLE II provides similar data for a specific medical instrument 130, namely an Olympus BF 2T10 flexible endoscope. When the motive pressure was set at about 349 KPa (KPa means kilopascals) and about 277 KPa, the suction pressure and suction flow rate had to be adjusted downward by adjusting valve 48 to reduce the bubbles in suction conduit 112. At about 138 KPa and about 70 KPa, valve 48 was completely open.

TABLE II

| MOTIVE | MOTIVE | | SUCTION |
|---|---|---|---|
| PRESSURE | FLOW RATE | SUCTION | FLOW |
| (×10⁵ Pascals- | (×10⁻² liters/ | PRESSURE | (×10⁻³ liters/ |
| gauge) | second) | (×10⁴ Pascals-gauge) | second) |
| 3.49 | 4.38 | −4.48 | 9.7 |
| 2.77 | 4.05 | −4.6 | 9.8 |
| 2.07 | 3.63 | −3.6 | 8.2 |
| 1.38 | 3.22 | −2.3 | 5.0 |
| 0.70 | 2.75 | −1.4 | 1.8 |

TABLE III provides similar data, where medical instrument 130 is a Karl Storz 27410SK rigid endoscope. When the motive pressure was set at about 345 KPa, the suction had to be reduced by adjusting valve 114 so as to reduce bubbles in suction conduit 112. At about 276 KPa, about 207 KPa, about 138 KPa and about 70 KPa, valve 114 was completely open. At about 70 KPa, there was no flow through suction conduit 112.

TABLE III

| MOTIVE | MOTIVE FLOW | | SUCTION |
|---|---|---|---|
| PRESSURE | RATE | SUCTION | FLOW |
| (×10⁵ Pascals- | (×10⁻² liters/ | PRESSURE | (×10⁻³ liters/ |
| gauge) | second) | (×10⁴ Pascals-gauge) | second) |
| 3.45 | 4.33 | −5.45 | 3.3 |
| 2.76 | 4.0 | −6.14 | 2.3 |
| 2.07 | 3.63 | −4.03 | 1.8 |
| 1.38 | 3.18 | −2.38 | 1.0 |
| 0.70 | 2.7 | −1.41 | 0 |

TABLE IV provides repeat data for a medical instrument 130, namely a Karl Storz 27410SK rigid endoscope. When the motive pressure was set at about 343 KPa and about 276 KPa, the suction had to be reduced by adjusting valve 114 so as to reduce bubbles in suction conduit 112. At about 207 KPa, valve 114 was completely open.

TABLE IV

| MOTIVE | MOTIVE FLOW | | SUCTION |
|---|---|---|---|
| PRESSURE | RATE | SUCTION | FLOW |
| (×10⁵ Pascals- | (×10⁻² liters/ | PRESSURE | (×10⁻³ liters/ |
| gauge) | second) | (×10⁴ Pascals-gauge) | second) |
| 3.43 | 4.35 | −5.45 | 2.3 |
| 2.76 | 4.05 | −6.14 | 2.2 |
| 2.07 | 3.62 | −4.0 | 1.3 |

As indicated above, bubbles in tubular portion 132 of medical instrument 130 and suction conduit 112 can be reduced by controlling the position of valve 114. The reduction of bubbles within washing fluid WF is thought to be beneficial as it is believed that, as the number of bubbles is reduced, more effective deactivation of biocontamination is achieved within the lumen of medical instrument 130.

It will be appreciated that any liquid can be pumped into inlet end 154 of eductor 150 to create suction at suction port 158 of eductor 150. In one embodiment, a deactivating fluid is pumped into inlet end 154 of eductor 150 to create suction at suction port 158 of eductor 150. Inasmuch as medical instrument 130, e.g., the endoscope, is submerged within the deactivating fluid within chamber 24, the exterior of medical instrument 130 and any biocontamination found on the exterior of medical instrument 130 may also be deactivated.

The foregoing description is a specific embodiment of the present invention. It should be appreciated that this embodiment is described for purposes of illustration only and that numerous alterations and modifications may be practiced by those skilled in the art without departing from the spirit and scope of the invention. It is intended that all such modifications and alterations be included insofar as they come within the scope of the invention as claimed or the equivalents thereof.

Having described the invention, the following is claimed:

1. A medical washer for deactivating surfaces of a medical instrument or device having a lumen, comprising:
a housing;
a chamber defined by said housing, wherein said chamber is dimensioned to contain a liquid and to receive a medical instrument having a lumen;
a primary conduit connected at both ends to said chamber to define a recirculating path for liquid in said chamber;
a suction device having an inlet port, an outlet port, and a suction port, wherein said suction device is disposed within said primary conduit such that said inlet port and said outlet port are fluidly connected to said primary conduit;
a suction conduit having a first end fluidly connectable to a lumen on a medical instrument and a second end fluidly connected to said suction port of said suction device; and
wherein said suction device is dimensioned such that a flow of said liquid through said suction device creates a negative pressure at said suction port.

2. A medical washer as defined in claim 1, wherein a pump is disposed within said primary conduit between said chamber and said inlet port of said suction device.

3. A medical washer as defined in claim 2, wherein a valve is disposed within said primary conduit between said pump and said suction device.

4. A medical washer as defined in claim 2, wherein a flow meter is disposed within said primary conduit between said pump and said suction device.

5. A medical washer as defined in claim 2, wherein a pressure indicator is disposed within said primary conduit between said pump and said suction device.

6. A medical washer as defined in claim 1, wherein a tank for containing said deactivating fluid is disposed within said primary conduit between said outlet port of said suction device and said chamber.

7. A medical washer as defined in claim 6, wherein a pump is disposed within said primary conduit between said tank and said chamber.

8. A medical washer as defined in claim 1, wherein a flow meter is disposed within said suction conduit.

9. A medical washer as defined in claim 1, wherein a pressure indicator is disposed within said suction conduit.

10. A medical washer as defined in claim 1, wherein a valve is disposed within said suction conduit.

11. A system to deactivate biocontamination within a lumen of a medical instrument, comprising:
a tank containing a deactivating fluid;
means for holding a medical instrument having a lumen immersed within the deactivating fluid;
a fluid flow path connected at both ends to said tank to define a closed-loop circulation system;
a pump for conveying said deactivating fluid along said closed-loop path;
an eductor disposed in said closed loop circulation system, said eductor having a fluid passage therethrough and a suction port, said eductor creating a negative pressure at said suction port when said deactivating fluid flows through said fluid passage; and
means for connecting the suction port of the eductor to one end of the lumen.

12. The system of claim 11, wherein a valve is disposed between the distal tip of the lumen and the suction port of the eductor.

13. The system of claim 12, further comprising an electronic controller.

14. A method of deactivating biocontamination found within a lumen of a medical instrument, the method comprising the steps of:
pumping a liquid through an inlet of an eductor thereby producing a suction at a suction port of the eductor, the suction port of the eductor fluidly connected to a distal tip of the lumen; and,
drawing a deactivating fluid that surrounds the medical instrument into a lumen inlet port, through the lumen and into the eductor through the suction port of the eductor by the suction produced within the eductor as the liquid is pumped through the inlet port of the eductor.

15. The method of claim 14, further comprising the step of:
controlling a valve disposed between the distal tip of the lumen and the suction port of the eductor to control the suction pressure and suction flow rate so as to reduce the number of bubbles within the deactivating fluid.

16. The method of claim 15, wherein the valve is controlled by an electronic controller.

17. The method of claim 14, wherein the liquid is the deactivating fluid.

18. The method of claim 14, wherein the deactivating fluid is reused.

19. The method of claim 14, wherein biocontamination found on the exterior of the medical instrument is deactivated.

20. A system for microbially deactivating a lumen in a medical instrument, said system comprising:
a housing defining a chamber for holding a deactivating fluid;
a closed-loop circulation system for circulating said deactivating fluid in said chamber, wherein said deactivating fluid is withdrawn from said chamber, conveyed along a closed-loop path and re-introduced into said chamber;
an eductor device having a fluid passage therethrough, said fluid passage having an inlet end, an outlet end, a flow restriction disposed between said inlet end and said outlet end, and a suction port communicating with said fluid passage between said inlet end and said flow restriction, said eductor being disposed in said closed-loop circulation system wherein said fluid passage forms a portion of said closed-loop path and said deactivating fluid flows through said eductor from said inlet end to said outlet end when said deactivating fluid is conveyed along said closed-loop path, said flow restriction in said eductor being dimensioned such that flow of deactivating fluid through said eductor creates a negative pressure at said suction port; and
a suction conduit connected at one end to said chamber and connectable at another end to a lumen in a medical instrument disposed in said chamber.

21. A system for microbially deactivating a lumen in a medical instrument as defined in claim 20, wherein said circulation system includes a first conduit that fluidly connects said chamber to said inlet end of said eductor; and
a second conduit that fluidly connects said outlet end of said eductor to said chamber.

22. A system for microbially deactivating a lumen in a medical instrument as defined in claim 21, wherein a pump is disposed within said first conduit.

23. A system for microbially deactivating a lumen in a medical instrument as defined in claim 22, wherein a valve is disposed within said first conduit.

24. A system for microbially deactivating a lumen in a medical instrument as defined in claim 23, wherein said valve is disposed within said first conduit between said pump and said inlet end of said eductor.

25. A system for microbially deactivating a lumen in a medical instrument as defined in claim 22, wherein a first flow meter is disposed within said first conduit between said pump and said eductor.

26. A system for microbially deactivating a lumen in a medical instrument as defined in claim 22, wherein a first pressure indicator is disposed within said first conduit between said pump and said eductor.

27. A system for microbially deactivating a lumen in a medical instrument as defined in claim 21, wherein a pump is disposed in said second conduit.

28. A system for microbially deactivating a lumen in a medical instrument as defined in claim 27, wherein a tank for containing said deactivating fluid is disposed within said second conduit between said outlet end of said eductor and said pump.

29. A system for microbially deactivating a lumen in a medical instrument as defined in claim 20, wherein a flow meter is disposed in said suction conduit.

30. A system for microbially deactivating a lumen in a medical instrument as defined in claim 20, wherein a pressure indicator is disposed in said suction conduit.

31. A system for microbially deactivating a lumen in a medical instrument as defined in claim 20, wherein a valve is disposed in said suction conduit.

32. A system for microbially deactivating a lumen in a medical instrument as defined in claim 20, wherein said suction conduit extends into said chamber.

33. A method for microbially deactivating a lumen in a medical instrument, the method comprising the steps of:
providing a closed loop path that is connected at both ends to a chamber for containing a deactivating fluid;
disposing a medical device having a lumen in said chamber such that an end of said lumen is fluidly connected to said closed loop circulation system;
conveying a first stream of said deactivating fluid along said closed-loop path, such that said first stream of said deactivating fluid creates a negative pressure within a first portion of said closed-loop path;
drawing a second stream of said deactivating fluid along a second path from said chamber such that said deactivating fluid is conveyed through said lumen, wherein said second path joins said closed-loop path at said first portion of said closed-loop path;
combining said first stream of deactivating fluid and said second stream of deactivating fluid at said first portion of said closed-loop path to form a combined stream of said deactivating fluid; and
reintroducing said combined stream of deactivating fluid into said chamber.

34. A method for microbially deactivating a lumen in a medical instrument as defined in claim 33, wherein said negative pressure is created hydraulically by said first stream.

35. A method for microbially deactivating a lumen in a medical instrument as defined in claim 33, wherein said negative pressure results from hydraulic action of said first stream within said first portion of said closed-loop path.

36. A method for microbially deactivating a lumen in a medical instrument as defined in claim 35, wherein an eductor having an inlet and an outlet is disposed within said closed-loop path and said eductor defines said first portion of said closed-loop path.

37. A method for microbially deactivating a lumen in a medical instrument as defined in claim 36, wherein a pump is disposed within said closed-loop path between said chamber and said inlet of said eductor.

38. A method for microbially deactivating a lumen in a medical instrument as defined in claim 37, wherein a first valve is disposed within said closed-loop path between said pump and said eductor, further comprising the step of:
adjusting said first valve such that bubbles are not present in said deactivating fluid as said deactivating fluid passes through said lumen.

39. A method for microbially deactivating a lumen in a medical instrument as defined in claim 37, wherein said second stream is conveyed through a second conduit having a second valve disposed therein, further comprising the step of:
adjusting said second valve such that bubbles are not present in said deactivating fluid as said deactivating fluid passes through said lumen.
